(19) United States
(12) Patent Application Publication
(10) Pub. No.: US 2018/0000432 A1
(43) Pub. Date: Jan. 4, 2018

(54) FLEXIBLE RADIOPAQUE APRON

(71) Applicant: Carestream Health, Inc., Rochester, NY (US)

(72) Inventor: Adam D. Pruyne, Webster, NY (US)

(21) Appl. No.: 15/371,510

(22) Filed: Dec. 7, 2016

Related U.S. Application Data
(60) Provisional application No. 62/356,122, filed on Jun. 29, 2016.

(51) Int. Cl. A61B 6/10; A61B 6/03; A61B 6/00; G21F 3/03

(52) U.S. Cl. CPC A61B 6/107; G21F 3/03; A61B 6/032; A61B 6/4085

(57) ABSTRACT

A radiopaque apron configured to overlap an opening of a radiographic imaging apparatus. The apron is a two-layered radiopaque flexible material that extends over the opening. A first layer is large enough to extend across the imaging opening. The second layer has an apron opening smaller than the imaging opening and is positioned between the first layer and the apparatus.

### CROSS REFERENCE TO RELATED APPLICATIONS

This application claims priority to U.S. Patent Application Ser. No. 62/356,122, filed Jun. 29, 2016, in the name of Adam D. Pruyne, and entitled RADIOPAQUE APRON.

This application is related in certain respects to U.S. Patent Application Publication U.S. 2014/0098930 A1, filed Oct. 8, 2013, in the name of Litzenberger et al., and entitled EXTREMITY IMAGING APPARATUS FOR CONE BEAM COMPUTED TOMOGRAPHY, which is incorporated herein by reference in its entirety.

### BACKGROUND OF THE INVENTION

The invention relates generally to diagnostic imaging and in particular to a cone beam radiographic imaging system used for obtaining volume images of patient extremities.

3-D radiographic volume imaging has proved to be a valuable diagnostic tool that offers significant advantages over earlier 2-D radiographic imaging techniques for evaluating the condition of internal structures and organs. 3-D imaging of a patient or other subject has been made possible by a number of advancements, including the development of high-speed imaging detectors, such as digital radiography (DR) detectors that enable multiple images to be taken in rapid succession.

Cone beam computed tomography (CBCT) technology offers considerable promise as one type of diagnostic tool for providing 3-D radiographic volume images. CBCT systems capture volumetric data sets by using a high frame rate digital radiography (DR) detector and an x-ray source, typically affixed to a gantry that rotates about the object to be imaged, directing, from various points along its orbit around the subject, a divergent cone beam of x-rays toward the subject. The CBCT system captures projections throughout the rotation, for example, one 2-D projection image at every degree of rotation. The projections are then reconstructed into a 3D volume image using various techniques such as filtered back projection approaches.

The CBCT apparatus captures a series of 2D projection images of a patient extremity placed in the imaging bore of the apparatus. A central axis of the rotating source and detector may also define a central axis of the imaging bore. A housing of the CBCT imaging system may be shaped to surround a cylindrical imaging bore.

In summary, for extremity imaging, one improvement may provide x-ray shielding proximate the imaging bore when a patient extremity is placed therein. Improved radiographic energy shielding provides acceptable radiation scatter levels throughout the scanning or imaging sequence, i.e., during the activation of the x-ray source and its rotation about the central axis.

A well designed radiopaque apron allows imaging a patient's extremity with the patient standing or seated comfortably and protected from radiographic energy leakage (scatter). The radiopaque apron may allow the capability to adjust the angle of the central rotational axis to suit patient positioning requirements, thereby improving patient accessibility, so that the patient does not need to contort, twist, or unduly stress limbs or joints that may have been injured in order to provide images of those extremities. The patient may stand or sit with normal posture, for example.

As described herein, the imaging bore of the CBCT apparatus allows a single imaging apparatus to be configurable for imaging any of a number of extremities, including knee, ankle, toe, hand, elbow, and other extremities. This also includes the capability to operate the imaging system in different imaging modes, including CBCT, two-dimensional (2-D) projection radiography, fluoroscopy, and other tomography modes. The imaging bore size is sufficiently large to accommodate an extremity of a patient but not the body of the patient. As an example, the imaging bore may not be large enough to accommodate a human torso or two patient extremities simultaneously.

In summary, the capability for straightforward configuration and positioning of the imaging apparatus allows the advantages of CBCT imaging to be adaptable for use with a range of extremities, to obtain volume images under a suitable imaging modality, with the image extremity presented at a suitable orientation under both load-bearing and non-load-bearing conditions, and with the patient appropriately standing or seated and protected from unneeded or excessive radiation.

### BRIEF DESCRIPTION OF THE INVENTION

A radiopaque apron is configured to overlap an opening of a radiographic imaging apparatus. The apron is a two-layered radiopaque flexible material that extends over the opening. A first layer is large enough to extend across the imaging opening. The second layer has an apron opening smaller than the imaging opening and is positioned between the first layer and the apparatus.

In one embodiment, an imaging apparatus having a housing includes an imaging opening in the housing for positioning a patient anatomy to be imaged. A radiopaque apron attached to the apparatus is used to cover the opening. The radiopaque apron may have a first layer extending continuously over the opening and a second layers disposed between the first layer and the apparatus, wherein the second layer has an opening centered proximate a central axis of the imaging opening to receive the patient anatomy.

In one embodiment, a radiopaque apron is configured to cover an imaging opening of an imaging bore of a CBCT imaging apparatus. The apron may include a first continuous layer large enough to extend across the imaging opening, and a second layer adjacent the first layer between the first layer and the imaging apparatus. An opening in the second layer smaller than the imaging opening may be centered on a central axis of the imaging opening.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIGS. 1A-1B are exploded views of a radiopaque apron;

FIG. 2 is a close-up view of a portion of FIG. 1B;

FIGS. 3A-3B show a cross-section and cross-section perspective view, respectively, of the first and second layers of the apron and attachment components;

FIGS. 4A-4C illustrates the radiopaque apron of the present disclosure in front, side, and rear views, respectively;

FIG. 5A shows a top view of a CBCT imaging apparatus;

FIG. 5B shows a perspective view of a CBCT imaging apparatus with an attached radiopaque apron shown in transparent view; and

FIGS. 6A-6D are images of the apron in various positions over an imaging opening of a CBCT imaging apparatus.

### DETAILED DESCRIPTION OF THE INVENTION

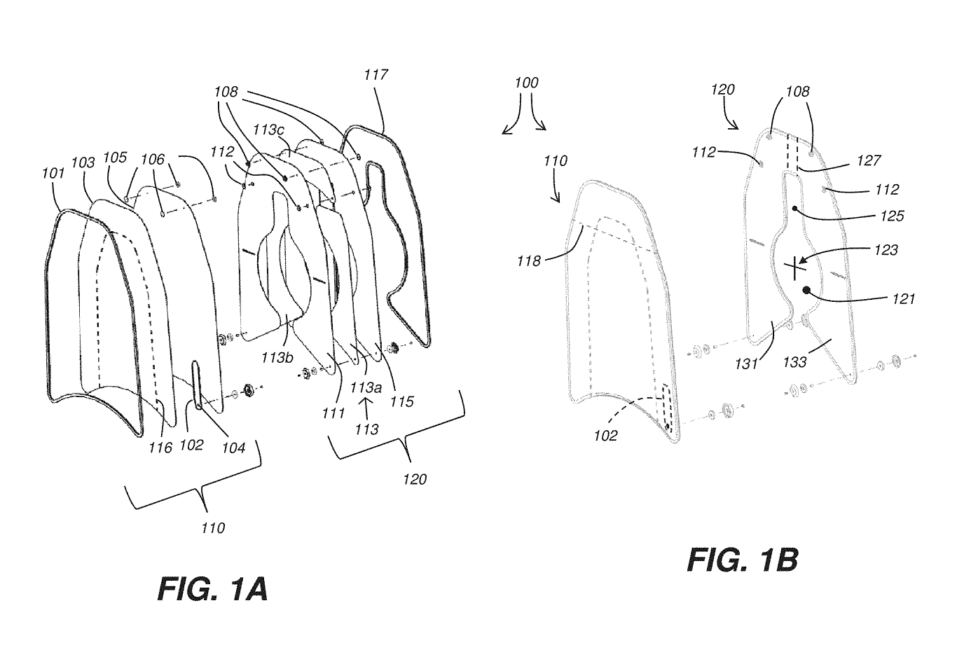

FIG. 1A illustrates the flexible radiopaque apron 100 of the present disclosure in an exploded view showing a flexible first layer 110 of the apron 100 comprising flexible sublayers 103, 105 and a flexible border, or edge, piece 101. A flexible second layer 120 of the apron 100 comprising flexible sublayers 111, 113, 115 and a flexible border, or edge, piece 117. FIG. 1B shows the first layer 110 and the second layer 120 as they would appear when their respective sublayers 103, 105, and 111, 113, 115, are assembled together, with their border pieces 101, 117, respectively. As shown, the first layer 110 may be said to comprise a substantially continuous, planar form while the second layer 120 may be said to comprise a central apron opening, or apron gap, 121, which central apron opening 121 may include a center point or area 123 illustrated by the symbol +. The central apron opening 121 may include an opening extension 125 extending away from a center 123 of the central apron opening 121 toward a top portion of the second layer 120. In one embodiment, the central apron opening, or apron gap, 121 is not completely enclosed, or surrounded, by the second layer 120. Rather, the second layer 120 comprises opposing flap portions 131, 133, that surround a major portion of the central apron opening 121 and almost converge, but do not come together in contact, at a bottom of the central apron opening 121.

In one embodiment, the second layer 120 may be assembled in a one-piece unitary fashion, or, in another alternative embodiment, it may be formed as a two-piece layer having two substantially symmetrical portions separated by a gap 127 at a top of the second layer 120. In the alternative embodiment, the sublayers 111, 113, 115, and the border piece 117 may each be similarly formed in two pieces before final assembly, or the sublayers 111, 113, 115, and the border piece 117 may be cut after assembly and then reassembled in the two-piece embodiment. The border pieces 101, 117, may be formed in the same material as one of their corresponding sublayers, or they may be formed using a different material. In one embodiment, the border pieces 101, 117, include a nylon material attached to their corresponding sublayers by being sewn through their corresponding sublayers. The border pieces 101, 117, may also be clamped, glued, or otherwise attached to their respective sublayers to continuously surround an edge of the first and second layers 110, 120.

As shown in FIG. 1A, the sublayer 103 may be formed from a flexible plastic, resin, polyvinyl, rubber, fabric, or a combination thereof, such as a Naugahyde sublayer, and may be designed and used for aesthetic purposes. The layer 105 may contain lead as a radiopaque material or it may include a sheet of material sold as Greenlite manufactured by Infab Corporation of Camarillo, Calif. The sublayer 103 may also contain a radiopaque coating or layer on its side that faces sublayer 105. In one embodiment, the layers 103, 105, may be stitched together such as along exemplary stitch line 116. As shown in FIG. 1A, the sublayers 111, 115 may be formed from a flexible plastic, resin, polyvinyl, rubber, fabric, or a combination thereof, such as Naugahyde sublayers, and may be designed and used for aesthetic purposes. The sublayers 111, 115, may contain lead as a radiopaque material or they may each include a sheet of material sold as Greenlite manufactured by Infab Corporation of Camarillo, Calif. The sublayers 111, 115, may also contain a radiopaque coating or layer on their sides that face each other. A sublayer 113 may be included in layer 120 to serve as a stiffener, made from a thin plastic, resin, or rubber material. In one embodiment, the individual pieces 113a-c may be selectively formed from a thin polycarbonate, or from a thermoplastic acrylic-polyvinyl chloride material commercially known as Kydex, for example. The sublayer 113 may still be flexible but slightly stiffer than layers 111 and 115. The sublayer 113 may be formed in one piece, two pieces, or, as shown in FIG. 1A, in three pieces 113a, 113b, and 113c.

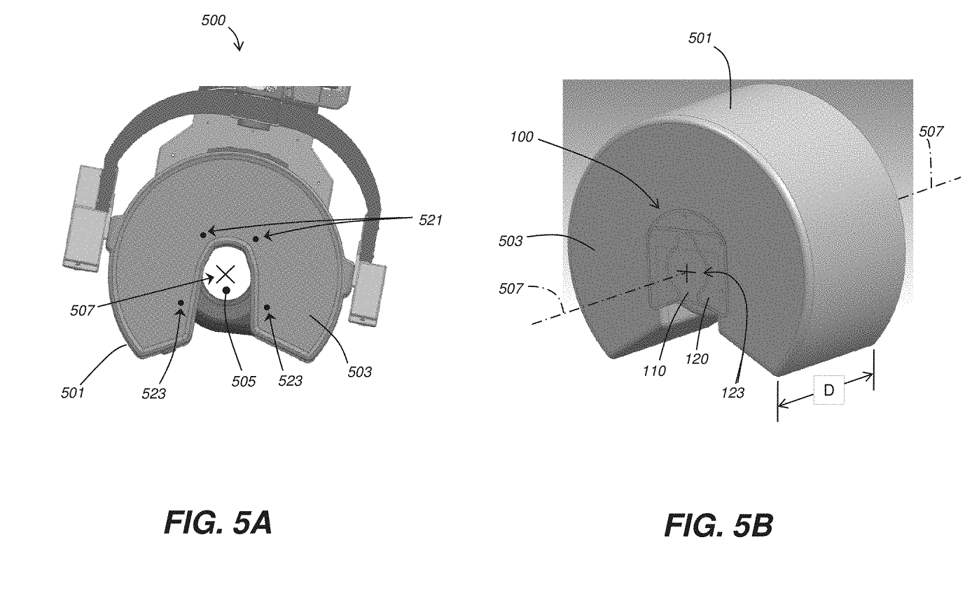

Layer 110 may include a pair of attachment elements, such as snaps 106 fixed in sublayer 105 by attaching together separate parts of the attachment elements 106 through holes in the sublayer 105. Attachment elements, such as snaps, 108, that mate with attachment elements 106 may be fixed in layer 120 by attaching together separate parts of the attachment elements 108 through holes in the sublayers 111, 113, 115. The attachment elements 106 may be connected to attachment elements 108 such as by being detachably attached together in the form of snaps. Layer 120 may further include grommets 112 that pass through holes in the sublayers 111, 113, 115. The grommets 112 allow the layer 120 to be attached to a housing of a CBCT imaging apparatus (FIG. 5A), using screws as described herein. Alternative embodiments of removable attachment elements 106, 108 may include hook and loop fastening material commonly sold as Velcro, magnetic segments, or other suitable attachment embodiments.

Layer 110 may include a pair of thin metal pieces 102 (only one of which is visible in the view of FIG. 1A) each having a threaded hole 104 therethrough that is aligned with a hole through sublayer 105, the assembly of which is described hereinbelow. The metal pieces 102 may be positioned between sublayers 103 and 105, and between stitches 116 and the border piece 101 when the layer 110 is fully assembled. Another stitch line 118 (FIG. 1B) may be sewn to secure together both layers 110, 120. As described below, the layer 110 may be flapped open using the stitch line 118 as a hinged line.

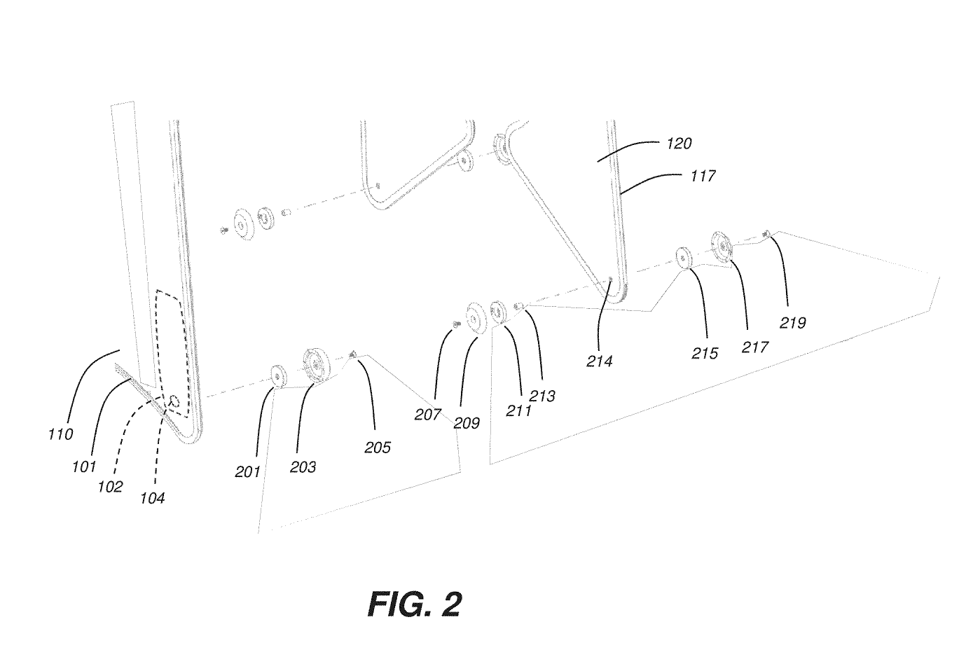

FIG. 2 is a close-up view of FIG. 1B showing exemplary attachment components for securing together the sublayers 103, 105, of layer 110 and the sublayers 111, 113, 115, of layer 120. The enumerated attachment components in FIG. 2 are described with respect to only one assembly thereof, but the enumerated attachment components are duplicated proximate a second opposite edge of the layers 110, 120 positioned in approximately a symmetric location with respect to the enumerated components' location, as shown hereinbelow.

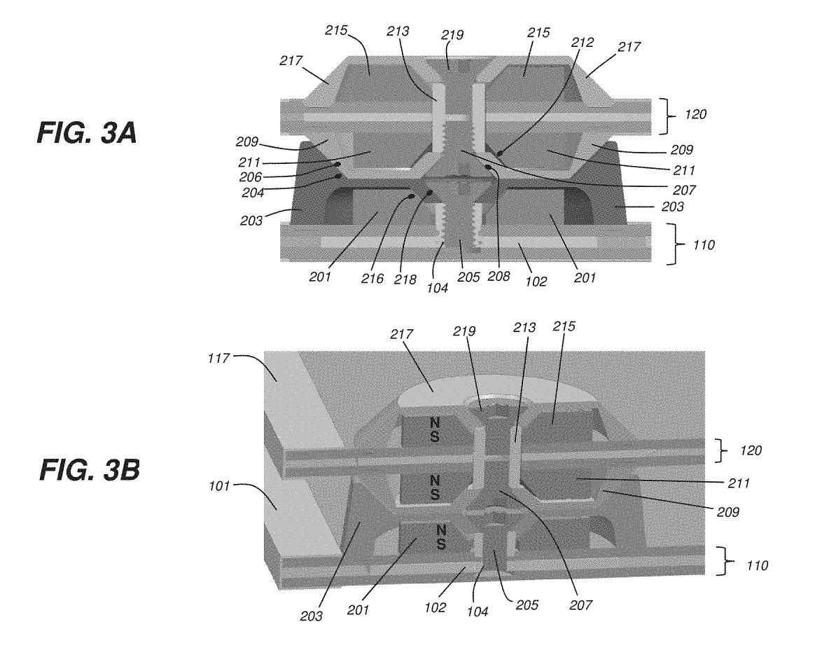

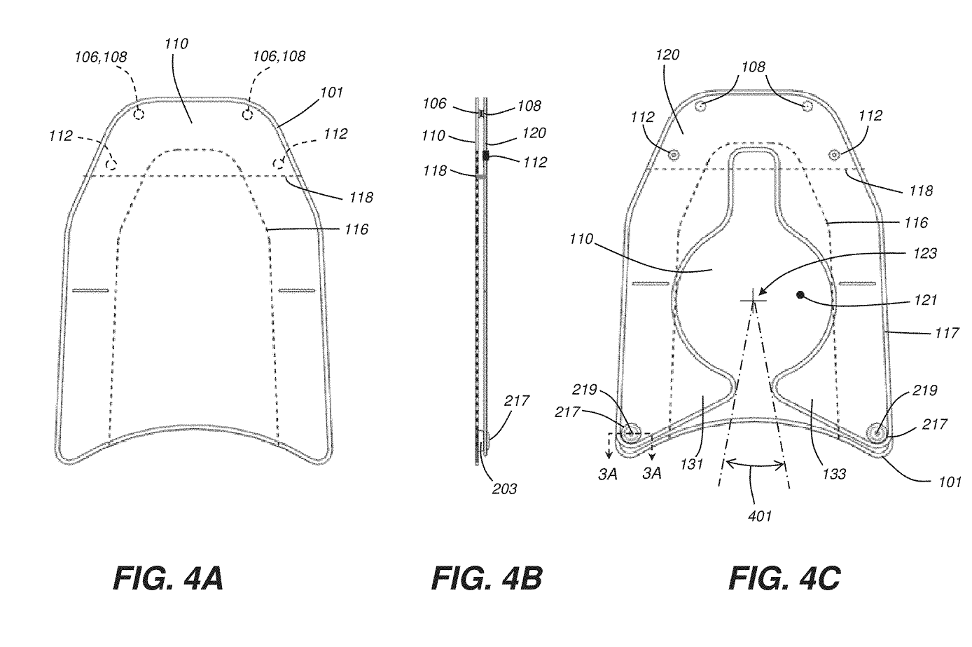

The following description may best be understood with reference to FIGS. 2 and 3A-3B. FIG. 3A shows a cross-section view of the attachment components fully assembled to layers 110, 120, as illustrated in FIG. 4C. FIG. 3B shows a perspective view of the cross-section view of FIG. 3A. A somewhat circular, or coin shaped, magnet 201 includes a hole therethrough to receive a screw 205. A cover, or cap, 203 includes a hole therethrough to receive the screw 205, and is shaped on one side to partially enclose the magnet 201. Both the magnet 201 and the cap 203 each include an annular frustoconical shaped countersink 216, 218, respectively, matching an angle of the head of the screw 205. The screw 205 screws into the threaded hole 104 of the metal piece 102 through a hole in sublayer 105 after passing through the holes in the magnet 201 and the cap 203. In this fashion, the assembly of the attachment components for the layer 110 is complete.

The cap 203 includes a further annular frustoconical countersink 204 to receive an annular frustoconical extension 206 of the cap 209. The cap 209 includes an annular frustoconical countersink 208 and hole therethrough to receive screw 207, and is configured to partially enclose somewhat circular, or coin shaped, magnet 211 which also includes an annular frustoconical countersink 212 and hole therethrough to receive the screw 207. Both annular frustoconical shaped countersinks 208, 212, match an angle of the head of the screw 207. A symmetrical arrangement of another cap 217, magnet, 215, and screw 219, is configured to be joined together in a similar fashion on an opposite side of layer 120 aligned with the assembly of cap 209, magnet 211, and screw 207. The cap 217 also includes a countersink hole therethrough to receive screw 219, and partially encloses magnet 217 which also includes a countersink hole therethrough to receive screw 219. Both the magnet 215 and the cap 217 each include an annular frustoconical shaped countersink (not enumerated) matching an angle of the head of the screw 219. A cylindrical element 213 having interior threads passes through a hole 214 in the sublayers of layer 120 and through the holes of magnets 211, 215. The screw 207 passes through the hole of cap 209 and the hole of magnet 211 and may be screwed into a first end of cylindrical element 213 from a first direction. The screw 219 passes through the hole of cap 217 and the hole of magnet 215 and may be screwed into a second end of cylindrical element 213 from a second direction. In this fashion, the assembly of the attachment components for layer 120 is complete. The magnets 201, 211, 215, interact in an attractive complementary fashion by arranging their polarities as shown by the North and South magnetic pole designations N and S in FIG. 3B, which arrangement may be reversed with equal effect.

The completed assembly of layers 110, 120, allows the top portions of the layers 110, 120, to be attached together using exemplary pairs of mating snaps 106, 108 (FIG. 4B). Similarly, the bottom portions of the layers 110, 120, may be attached together using the mating caps 203, 209, which are drawn together by magnetic attraction generated at least by magnets 201, 211. Alternative embodiments of attachment components usable to attach the bottom portions of the layers 110, 120, may also include hook and loop fastening material commonly sold as Velcro, snaps, or other suitable attachment embodiments.

Referring again to FIGS. 1A-1B, the border pieces 101, 117, may be attached to their respective sublayers before or after the sublayers are secured together. In one embodiment, the border pieces 101, 117, may be attached to their respective sublayers simultaneously with attaching the sublayers together such as by sewing the border pieces through their corresponding sublayers. Although the first layer 110 and second layer 120 are shown in an exemplary embodiment as formed from two or more sublayers, the first and second layers 110, 120, may be formed from only one layer or from more than three layers, as desired. In a one-sublayer embodiment, the one-sublayer may be impregnated, coated, deposited, or otherwise combined with a radiopaque material. The sublayers 101, 103, and 111, 113, 115, may themselves be secured together using a sewing machine as described herein, or they may be secured together using staples, rivets, or other known components used for securing together materials in the form of sheets. The sublayers 101, 103, and 111, 113, 115, may be laminated together, or they may be coated with adhesive on their surfaces that face each other and compressed, heated, ultraviolet light (UV) exposed, or otherwise flexibly attached, as desired.

FIGS. 4A-4C illustrate the radiopaque apron 100 of the present disclosure in a front view, side view, and rear view, respectively, with many of the elements described herein enumerated. FIG. 4A shows the position of the stitch line 118 which may be used to secure together the layers 110 and 120 such as by machine sewing. Stitch line 116 may be used to sew together the sublayers of layer 110. Exemplary positioning of the attachment elements 106 of layer 110 are illustrated in FIG. 4A, which may be used to attach together the upper portions of the layers 110 and 120 using cooperative attachment elements 108 in layer 120, as shown in FIGS. 4B and 4C. The nesting fit as between mating attachment components, or caps, 203, 209, is illustrated in the side view of FIG. 4B, wherein cap 209 is not visible due to its position within the frustoconical countersink 218 of cap 203. Cap 209 has a similar profile as that shown by cap 217 in FIG. 4B, however, cap 209 extends from the opposite side of layer 120 into mating cap 203.

As shown in FIG. 4C, the apron opening 121 in layer 120 may be said to have a center 123, as illustrated by the + symbol. The lower portion of layer 120 includes two flaps 131, 133, separated by a gap which may be measured by the angle 401 delineated by the center 123 and the interior edges of the flaps 131, 133. The angle of the gap may range from a small angle of about 5° to a large angle of over 90° . As shown in FIG. 4C, angle 401 is slightly more than about 20°. Thus, with an exemplary gap angle 401 of about 20°, the layer 120 may be said to surround the apron opening 121 for about 340°; or with an exemplary gap angle 401 of about 90°, the layer 120 may be said to surround the apron opening 121 for about 270°, for example. In one preferred embodiment, the layer 120 surrounds the apron opening 121 for at least about 180°.

FIG. 5A shows a top view of an exemplary CBCT imaging apparatus 500, such as described in the Patent Application Publication U.S. 2014/0098930 A1 identified above and incorporated herein by reference. FIG. 5B shows a perspective view of the housing 501 portion of FIG. 5A. The CBCT imaging apparatus 500 comprises a C-shaped housing 501 enclosing radiographic source and detector components for imaging patient anatomies that are positioned in the imaging opening 505 proximate a central axis 507, indicated by the symbol ×, of the imaging opening 505. FIG. 5A shows the imaging apparatus 500 and the imaging opening 505 uncovered by the radiopaque apron 100. In a top view, as shown in FIG. 5A, the imaging opening 505 may be said to have an approximate circular shape, or at least a portion of the opening 505 may be shaped as an arc of a circle. The imaging opening 505 extends through an entire depth D (FIG. 5B) of the housing 501 to form an imaging volume that may be referred to herein as an imaging bore. The imaging bore formed by the housing 501 defines a somewhat cylindrical imaging volume wherein a patient anatomy may be positioned for radiographic imaging. The top surface 503 of the housing 501 (facing the reader in FIG. 5A) and bottom surface (not shown), opposite the top surface, form two major surfaces of the housing 501. An interior circumferential housing surface extends between and connects the top and bottom surfaces thereby forming the somewhat cylindrical imaging bore surrounding the central axis 507. The radiation source and detector each may orbit a patient anatomy positioned in the imaging bore at the central axis 507 to obtain various radiographic images of the patient anatomy. Portions of the housing 501 may be fabricated with metal, fiberglass, plastic, or other suitably rigid material. According to one embodiment, all of, or portions of, the top surface 503 and the opposite bottom surface (not shown) are substantially flat.

FIG. 5B illustrates the radiopaque apron 100 of the present disclosure attached to the top surface 503 of the housing 501. The apron 100 is shown in a transparent view, wherein an outline of the second layer 120 can be seen beneath the first layer 110 that covers it (see e.g., FIG. 4A). A center 123 of the layer 120 apron opening 121 is positioned over the imaging opening 505 such that it is proximate to or coincides with the central axis 507 of the imaging opening 505. Attachment components 521, 523, for attaching the radiopaque apron 100 to a top surface 503 of the housing 501 are shown in FIG. 5A. Attachment components 521 may include threaded holes for receiving screws that pass through grommets 112 in layer 120 of the radiopaque apron 100. Thus, the radiopaque apron 100 may be somewhat permanently attached to the housing 501 of imaging apparatus 500 depending on the type of screws used. In one embodiment, the apron 100 may not be detached from the housing 501 without the use of at least one tool. Attachment components 523 may include caps that magnetically mate with the caps 217 of the radiopaque apron 100. In one embodiment, attachment components 523 may each include a magnet and a cap each having a hole therethrough, and a screw to attach the assembly to a threaded hole in the top surface 503 of the housing 501. Such an assembly of magnet, cap, and screw may resemble the magnet 201, the cap 203, and the screw 205 shown in FIG. 2. Because the cap 217 on layer 120 resembles the cap 209 which nests within the cap 203, the cap 217 would also nest into a cap positioned as an attachment component 523 having a similar or identical construction as cap 203. The magnet within the housing attached cap would be oriented to attract the magnet 215 thereto, thereby securing layer 120 to the top surface 503 of the housing 501 by magnetic force. In turn, layer 110 may be secured to the layer 120 by magnetic force, as described herein.

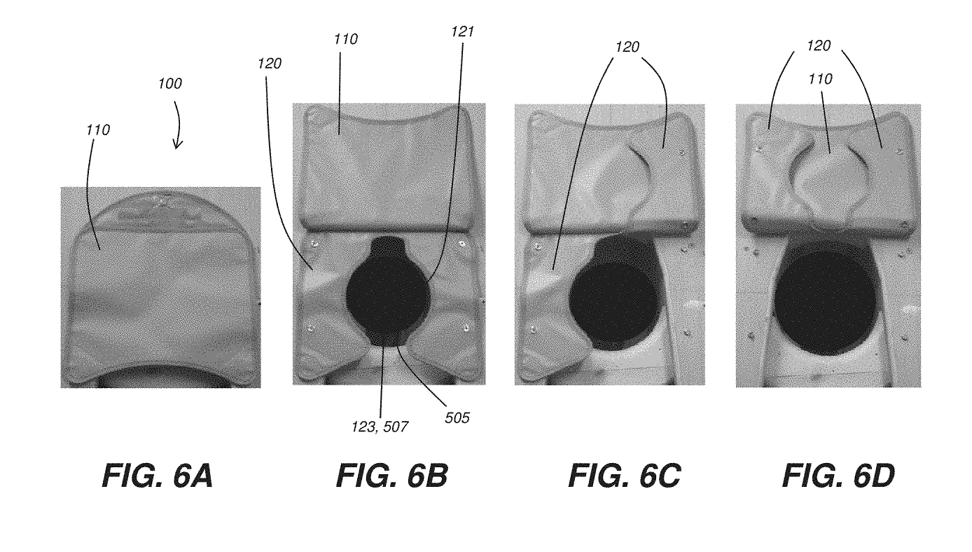

FIGS. 6A-6D illustrate the radiopaque apron 100 of the present disclosure attached to a housing of an imaging system (FIG. 6A); with the first layer 110 folded up and the second layer 120 extending over the edges of the imaging opening 505 (FIG. 6B). A center of the apron opening (+) 123 may be positioned proximate a central axis (×) 507 of the imaging opening 505 as shown in FIG. 6B. Either or both flaps of the second layer 120 may also be folded up, as desired, as shown in FIGS. 6C-6D.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An imaging apparatus comprising:
a housing;
an imaging opening in the housing for positioning a patient anatomy to be imaged; and
a radiopaque apron attached to the apparatus proximate the imaging opening,
wherein the radiopaque apron comprises two overlapping flexible layers, a first one of the layers extends continuously over the imaging opening, a second one of the layers is disposed between the first layer and the apparatus, and wherein the second layer comprises an apron opening centered proximate a central axis of the imaging opening.

2. The apparatus of claim 1, wherein the apron is attached to a housing of the apparatus proximate the imaging opening by a plurality of attachment elements that penetrate a portion of the apron and are affixed to the housing.

3. The apparatus of claim 2, wherein the radiopaque apron comprises lead (Pb).

4. The apparatus of claim 2, wherein the second layer overlaps an edge of the housing at the imaging opening and extends around the imaging opening for greater than about 180° but not entirely around the imaging opening.

5. The apparatus of claim 1, wherein the imaging opening comprises a central axis, and wherein the second layer is configured to flexibly contact an extremity of a patient at least on diametrically opposite sides of the extremity relative to the central axis of the imaging opening.

6. The apparatus of claim 5, wherein the second layer is configured to flexibly contact an extremity of a patient while substantially covering a gap between the extremity of the patient and the edge of the opening.

7. The apparatus of claim 1, wherein the first layer comprises a first magnet, the second layer comprises a second magnet, and wherein the first and second magnets are configured to hold together the first and second layers by a magnetic force.

8. The apparatus of claim 7, wherein the housing comprises a third magnet, and wherein the third magnet is configured to attract the second magnet against the third magnet.

9. The apparatus of claim 7, wherein the first magnet is secured within a first mating element and the second magnet is secured within a second mating element such that the first and second mating elements fit together to maintain a preselected orientation therebetween.

10. The apparatus of claim 9, wherein the housing comprises a third magnet, the third magnet is configured to attract the second magnet against the third magnet, the second magnet is secured within an apron mating element and the third magnet is secured within a housing mating element such that the apron mating element and the housing mating element fit together to maintain a preselected orientation therebetween.

11. A radiopaque apron configured to cover an imaging opening of an imaging bore of a CBCT imaging apparatus, the radiopaque apron comprising:
a first continuous layer large enough to extend across the imaging opening;
a second layer adjacent the first continuous layer, the second layer disposed between the first layer and the imaging apparatus, the second layer comprising an apron opening smaller than the imaging opening and centered on a central axis of the imaging opening.

12. The apron of claim 11, wherein the second layer is configured to be positioned around an edge of the imaging opening without completely covering the imaging opening.

13. The apron of claim 12, wherein the second layer comprises two sections each positioned about a portion of the edge of the opening each at diametrically opposed positions with respect to a central axis of the imaging opening.

14. The apron of claim 13, wherein the apron is attached to a housing of the apparatus proximate the imaging opening by a plurality of attachment elements that penetrate a portion of the apron and are affixed to the housing.

15. The apron of claim 14, wherein the second layer overlaps an edge of the housing at the imaging opening and extends around the imaging opening for at least 180° but not entirely around the imaging opening.

16. The apron of claim 15, wherein the imaging opening comprises a central axis, and wherein the second layer is configured to flexibly contact an extremity of a patient at least on diametrically opposite sides of the extremity relative to the central axis of the imaging opening.

17. The apparatus of claim 16, wherein the first layer comprises a first magnet, the second layer comprises a second magnet, and wherein the first and second magnets are configured to hold together the first and second layers by a magnetic force.

18. The apparatus of claim 17, wherein the housing comprises a third magnet, and wherein the third magnet is configured to attract the second magnet against the third magnet.

19. The apparatus of claim 18, wherein the first magnet is secured within a first mating element and the second magnet is secured within a second mating element such that the first and second mating elements fit together to maintain a preselected orientation therebetween.

20. The apparatus of claim 19, wherein the third magnet is secured within a third mating element such that the second mating element and the third mating element fit together to maintain a preselected orientation therebetween.
